(19) United States
(12) Patent Application Publication
(10) Pub. No.: US 2026/0104353 A1
(43) Pub. Date: Apr. 16, 2026

(54) Rapid phase length control in a cavity enhanced spectrometer using a wavelength monitor error signal

(71) Applicant: Picarro, Inc., Santa Clara, CA (US)

(72) Inventors: Chris W. Rella, Sunnyvale, CA (US); Sze Meng Tan, Santa Clara, CA (US); John A. Hoffnagle, San Jose, CA (US)

(21) Appl. No.: 18/912,489

(22) Filed: Oct. 10, 2024

(51) Int. Cl. G01N 21/31; G01N 33/00

(52) U.S. Cl. CPC G01N 21/31; G01N 33/0027; G01N 2201/06113

(57) ABSTRACT

Improved cavity enhanced optical spectroscopy is provided by appropriate per-mode averaging of measured vs. desired frequencies. By using this information to control the optical path length of the cavity, the frequency comb defined by the cavity at which measurements are performed can be substantially stabilized vs. perturbations such as changes in gas sample pressure.

### FIELD OF THE INVENTION

This invention relates to cavity-enhanced optical spectroscopy.

### BACKGROUND

Cavity enhanced optical spectroscopy is an important technique for increasing the sensitivity of spectroscopic measurements. Cavity ring-down spectroscopy (CRDS) is a representative example of such a technique, and is based on measuring the ring-down time of a mode in the optical cavity as a measurement of the absorption in the cavity. CRDS for gas spectroscopy involves placing a sample gas inside the optical cavity, and then performing absorption spectroscopy of that gas sample based on the ring-down measurements.

Thus CRDS necessarily amounts to making measurements at a discrete set of frequencies (i.e., the frequency comb defined by the optical cavity). In practice, this frequency comb cannot be assumed to be stable from one scan to the next. For example, the optical path length in the cavity can change relatively rapidly as the pressure and/or composition of the sample gas changes.

Although a wavelength monitor can be used to measure the frequencies at which ring-downs occur, there is the complication that in practice the wavelength monitor will have errors, and these errors can be frequency-dependent. Elimination of such errors in hardware, when possible, tends to require complex frequency control and locking that is incompatible with rapid gas spectroscopy measurements from an instrument of reasonable cost.

Accordingly, it would be an advance in the art to provide CRDS or the like with substantially enhanced stability of the frequency comb, without reliance on complex/expensive frequency control.

### SUMMARY

When performing CRDS, it is desirable for sequential scans to be at the same set of frequencies. As indicated above, two of the main obstacles to doing this are rapid changes in the optical path length of the cavity due to changes in sample gas pressure and/or composition, and measurement error in the wavelength monitor used to measure the frequencies at which ring-downs occurred.

The approach of this work is to maintain a set of statistics r[i], one for each ring-down mode i, that amount to a per-mode calibration for both of these issues. The free spectral range of the cavity is assumed to be fixed, so it is natural to regard both the frequency errors w[i] and the calibration statistics r[i] to be angular variables, confined to the unit circle.

At each ring-down, the transducer is adjusted to tend to drive a discrepancy between measured frequency difference w[i] and the per-mode statistic r[i] to zero. Also at each ring-down, the per-mode statistic r[i] is updated according to its current value and the current value of w[i], effectively making r[i] an average of w[i] with an effective time constant for the averaging.

To see how this works, let's consider a steady-state situation after a large number of ring-downs with no changes to the optical path length of the cavity. In this situation, each w[i] as it is measured would agree with the corresponding r[i], so no changes would be made to the transducer from one ring-down to the next. Here it is self-evident that successive scans will be at the same set of ring-down frequencies.

Now we consider the effect of an instantaneous change of the optical path length of the cavity (e.g., a sudden change in gas pressure) after achieving this steady state. It's clear that each of the w[i] will have a corresponding offset from its corresponding r[i], and that the transducer will be adjusted over several ring-downs to drive this difference to zero. The important point here is that the cavity mode frequencies before and after this perturbation are the same. The change to the physical path length caused by the transducer compensates for the assumed change in cavity refractive index.

In the real system, the time constants etc. are selected to approximate this behavior of the idealized situation, e.g., by having the time constant for updating r[i] be substantially longer than the time scale of optical path length changes due to changes in gas sample pressure and/or concentration.

### BRIEF DESCRIPTION OF THE DRAWINGS

FIG. 1 shows steps of an exemplary embodiment of the invention.

FIG. 2 shows an exemplary mode scanning pattern.

FIG. 3 shows an exemplary per-mode wavelength monitor error.

FIG. 4 shows time-dependence of frequency error per mode from a numerical example.

FIG. 5 shows time-dependence of reference statistic per mode from the numerical example.

FIG. 6 shows time-dependence of actuator adjustment per mode from the numerical example.

FIG. 7 shows time-dependence of averaged reference statistic from the numerical example.

FIG. 8 shows automatic compensation of a refractive index change (top) by cavity length adjustment (middle) to stabilize the frequency comb (bottom).

### DETAILED DESCRIPTION

### A) an Exemplary Algorithm

The context for this example is improved control of a CRDS instrument where tuning the laser is used to generate ring-downs. The length of the cavity is controlled by a piezo-electric transducer (PZT), which is moved sufficiently slowly that on successive ringdowns, the cavity modes may be assumed to be separated by a fixed free spectral range (FSR).

If the composition or pressure of the gas within the cavity changes, the change in refractive index shifts the comb of cavity mode frequencies, but the corresponding change in the FSR is usually small enough to be ignored. If the PZT is moved appropriately, it is possible to compensate for the effects of the change of the refractive index, restoring the original frequencies of the cavity modes. This approach provides a method for controlling the cavity length rapidly in real-time using measurements of the ringdown frequencies made by a wavelength monitor to derive the required motion of the PZT. A feature of the proposed method is its ability to cope with imperfections in the wavelength monitor measurements, so long that these remain stable on timescales which are long compared to those on which the refractive index of the gas is varying.

In order to achieve high-speed control of the cavity length, we wish to gain information on each ringdown that will allow us to update the position of the PZT. We target frequencies Fₜ[i] which are separated by integer multiples of the cavity FSR, and search for a cavity mode (indexed by i) which is close to each of these targets at which a ringdown can occur. Using the wavelength monitor to measure the frequency of the light when each ringdown occurs, we obtain the values fₘ[i]. The differences Δ[i]=fₘ[i]−Fₜ[i] give us information about the offset between the comb of cavity modes and the collection of target frequencies and these can be used to move the PZT so they are aligned (meaning that the offset is reduced to zero).

We define the difference between the measured and targeted frequencies normalized by the cavity FSR as

$${Δ}^{′} [ i ] = \frac{2 π Δ [ i ]}{FSR}$$

For the purpose of adjusting the PZT, it is convenient to regard Δ′[i] as defining an angle measured in radians. If we first assume that the wavelength monitor is perfect, so that each fₘ[i] coincides with a cavity mode frequency, every Δ′[i] tells us how far the comb of cavity modes is shifted from the grid of target frequencies due to the position of the PZT. We may use this to adjust the PZT and ultimately reduce the differences to zero. If the composition of the gas or the pressure were to change, affecting the refractive index, the grid of cavity modes will move, and the differences will become non-zero. We can use this information to move the position of the PZT so that the mode frequencies again coincide with the target frequencies.

Let us now consider the situation in which the wavelength monitor may have imperfections which cause it to measure the mode frequencies slightly incorrectly, the errors being possibly frequency dependent. There can also be a constant frequency offset affecting all modes, if the monitor measures relative rather than absolute frequency. If we assume that these errors change slowly with time and frequency compared to the timescale of variations in refractive index, it becomes possible to consider storing the collection of differences Δ′[i] for a multiplicity of cavity modes (indexed by i) collected under conditions when the refractive index is not changing, and use this as a reference. If during the course of data collection, the measured differences are found no longer to match these reference values, this is evidence which may be used to update the PZT position. Since information is still being obtained on each ringdown, these updates can be prompt, and the storage of reference values per mode allows wavelength monitor inaccuracies to be compensated, giving consistent update information independent of the order in which modes are being measured.

In practice, we prefer to update the reference values on a slow timescale as the data are collected, rather than determining them once while the refractive index is unchanging and fixing them thereafter. This approach allows for the gradual tracking of drifts and other systematic effects. In the following description of the algorithm, we find it convenient to use the techniques of directional statistics on a circle to capture the fact that the comb of cavity modes is unchanged whenever the cavity round-trip length is changed by an integer multiple of the optical wavelength.

Corresponding to the frequency differences between the measured and targeted frequencies Δ′[i], we define the complex numbers

$$w [ i ] = exp ( j {Δ}^{′} [ i ] )$$

where j=\sqrt{−1}. The reference values against which these are compared are stored in another set of complex numbers r[i], one for each mode at which a ringdown is collected. As mentioned above, these reference values should hold information about the frequency differences under conditions of unchanging refractive index, while the composition and pressure of the sample are stable. For the present, let us assume that the r[i] are given quantities as we shall describe how they are updated later.

On the fastest timescale of individual ringdowns, we calculate an error signal which is used to adjust the PZT from the discrepancy which is defined to be

$$δ [ i ] = [ arg ( w [ i ] ) - arg ( r [ i ] ) ] mod 2 π$$

where the reduction modulo 2π is into the interval [−π, π]. We control the motion of the PZT to drive this discrepancy towards zero by calculating the amount by which the PZT should be adjusted from its present value by using

$$PZT\_adjust = min ( M , max ( - M , ( pzt\_scale ) δ [ i ] ) )$$

The value of PZT_adjust is conveniently expressed in units of the distance required to change the cavity round-trip length by a wavelength. Its value is clamped to the range (−M,M) to limit the maximum adjustment on each step, and the pzt_scale factor controls the rate at which the discrepancy is driven towards zero.

Each time a ringdown is obtained on mode i, the corresponding reference value r[i] is updated using

$$r [ i ] ← K [ ( 1 - α ) r [ i ] + α w [ i ] ]$$

where w[i] is computed from the difference between measured and target frequencies for that ringdown. The exponential weighting factor α is calculated for each ringdown to give an effective time constant for the average. The calculation of a is facilitated by maintaining a table of timestamps at which r[i] was last updated. K is a correction factor described below.

When there is an update equation of the form X[i]*←(1−α)X[i]+αy[i] which is being calculated every T seconds, the effective time constant τ is related to α by

$$τ = \frac{- T}{log ( 1 - α )}$$

or equivalently

$$α = 1 - exp ( - T / τ )$$

The second equation here is useful for us to choose the appropriate value of α if we want to emulate a given physical time constant. In the case of updating r[i], we can keep track of when the i′th mode was last measured and use this to select a to give the desired effective time constant. In the case of updating R (as shown below), the time between updates is the average time between ringdowns.

It remains to define the value of K, which is a near-unity correction factor that is used to change the reference values r[i] gradually so that they have desirable properties. In the above, the reference values are said to store a pattern of frequency differences under conditions of unchanging cavity loss and refractive index. As described so far, if the frequency differences for all the modes were simultaneously perturbed by the same amount, there would be no “restoring force” to relax away that perturbation. Similarly, it is desirable to minimize the amount by which the PZT needs to be adjusted averaged over all the modes. These can be achieved by introducing (small) relaxation factors β and γ and setting

$$K = exp ( - j ( γ arg R - β δ [ i ] ) ) = exp ( - j ϵ )$$

where R is obtained via a slow exponential average (computed ringdown by ringdown) over the reference values

$$R ← ( 1 - ρ ) R + ρr [ i ] ,$$

the small quantity ρ being chosen to set the rate of the exponential average. Even though these corrections are applied on each ringdown (at mode i), their effects are slow and cumulative since ϵ is small. Another consequence of E being small is that K is near-unity, as indicated above.

The complex reference values r[i] are preferably initialized to small real quantities (typically given by the square root of the machine precision). This avoids arithmetic errors when computing the argument, and the small moduli indicates that we are initially uncertain about the values. The update rule above moves the points away from the origin and gives each of them an argument which is primarily determined by that of w[i]. If the values of w[i] for mode i are consistent over many measurements, r[i] will approach the point on the unit circle equal to the common value of these w[i].

The rate at which the reference quantities r[i] change should be slow compared to the timescale over which the refractive index of the sample changes due to composition and pressure changes. This is because r[i] is intended to encapsulate the properties of the system while the sample is unchanging. The primary update mechanism for r[i] is via the constant α, so the time constant associated with a is significant.

The rate at which changes to r[i] occur as a result of the correction factor K are more difficult to determine since the relaxation factors β and γ are used to make small incremental corrections, which are applied only when the i′th mode is measured, which is much slower that the rate at which the discrepancy δ[i] and R are themselves changing (essentially on the timescale of individual ringdowns). We adjust these relaxation factors until there is no evidence of instability in the motion of the PZT when transients such as pressure changes occur.

The following exemplary numbers will depend on the average ringdown rate and the times at which specific cavity modes are visited, so they may not always be appropriate:
α is chosen to give an effective time constant of 120 s (more generally, 60 s to 360 s),
ρ is set to 0.0001, corresponding to a time constant of about 50 s (more generally a time constant range from 30 s to 120 s),
β is set to 0.2 (more generally in a range from 0.05 to 0.5),
γ is set to 0.01, (more generally in a range from 0.002 to 0.05).

Alternatively, these preferred time scales can be defined in terms of T_{scan}, the time it takes to perform a single spectral scan over the frequency range of interest. Preferably, the time constant corresponding to α (i.e., τ_{α}) is in a range between 10 T_{scan} and 1000 T_{scan}. Preferably, the time constant corresponding to ρ (i.e., τ_{ρ}) is in a range between 10 T_{scan} and 100 T_{scan}.

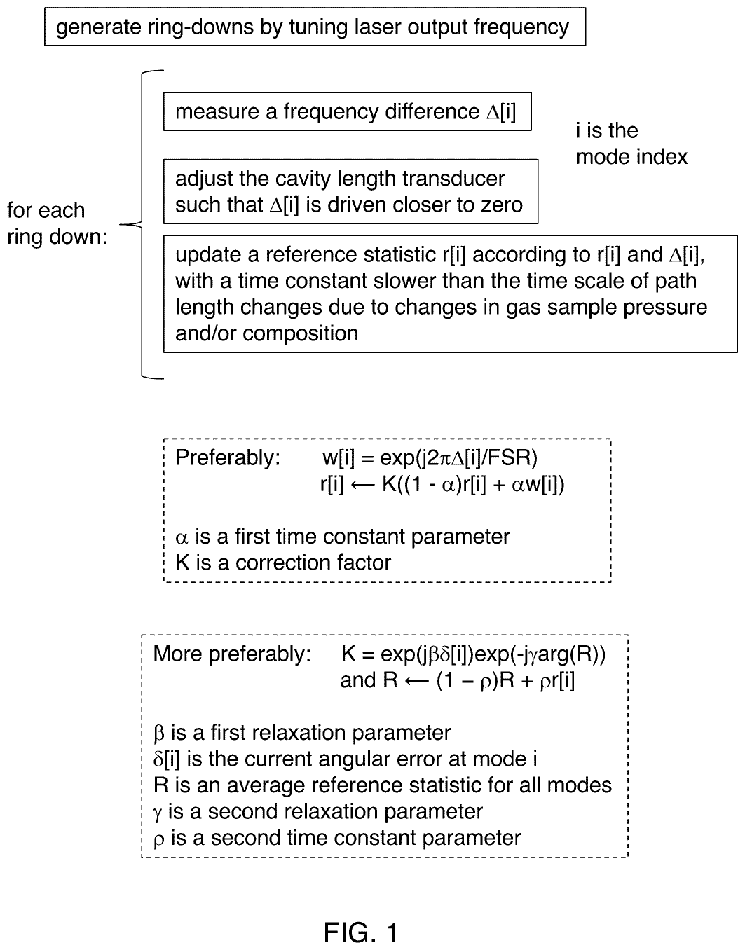

FIG. 1 schematically shows the steps of this method, as applied to CRDS. Here steps enclosed with dashed lines are optional, not required.

Although the preceding description has focused on CRDS, this idea of stabilizing the frequency comb provided by the cavity is more broadly applicable. In particular, it can be generalized to any cavity enhanced absorption spectroscopy (CEAS) that retains a clear longitudinal mode structure (i.e., not off-axis ICOS where the longitudinal mode structure of the cavity is deliberately suppressed, but other CEAS methods like optical-feedback CEAS).

Another variation is to vary the optical path length of the cavity by varying refractive index (e.g., by varying gas pressure), instead of varying the cavity length with a PZT or the like.

### B) Numerical Examples

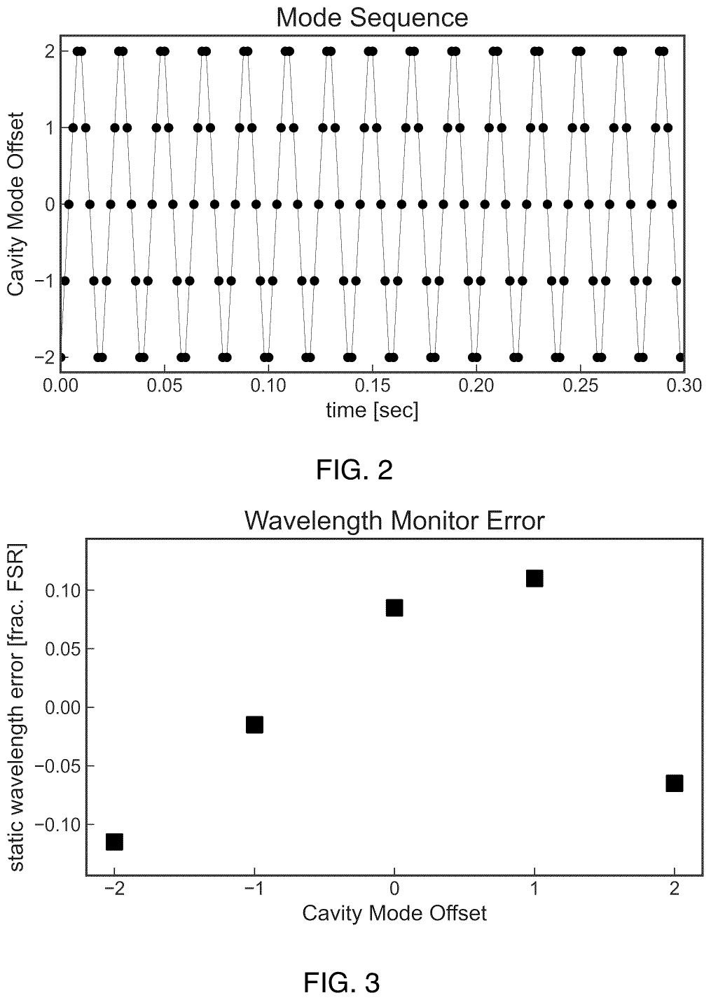

FIGS. 2-8 relate to numerical simulations performed to demonstrate operation of the above-described method.

FIG. 2 shows repeated spectral scanning over a set of 5 cavity modes. In practice, the number of relevant modes is typically much larger than 5, but we limit the number of modes here to 5 to make the underlying principles more clear.

FIG. 3 shows the static error assumed for the wavelength monitor for each of the 5 modes. Here this error is mode-dependent.

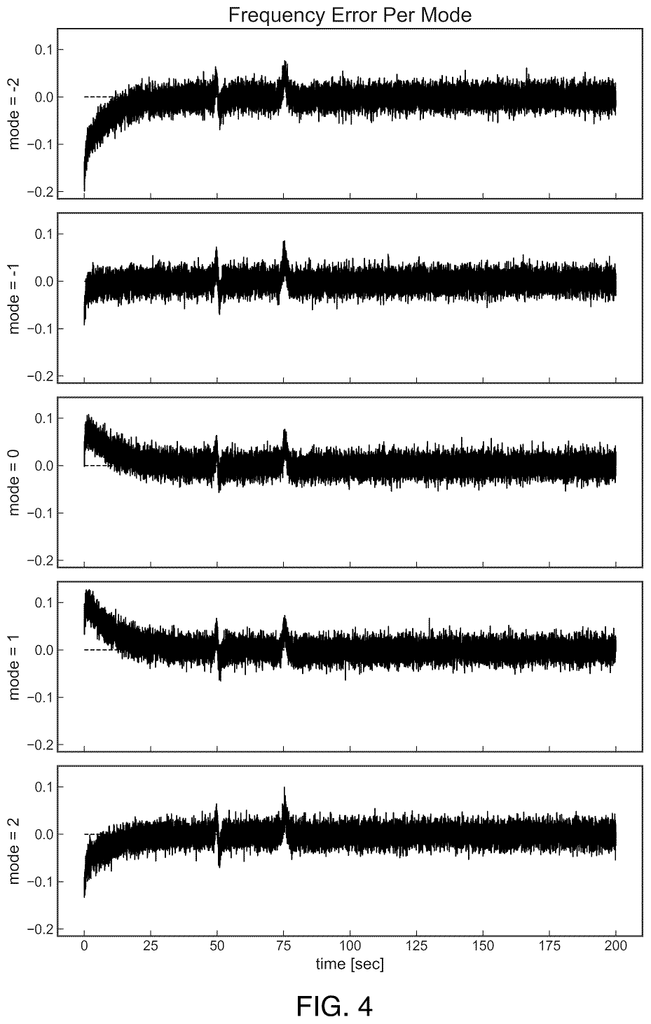

FIG. 4 shows simulation results of the measured error at each mode while the above-described method is operational. After an initial transient, the results are stable except at 50 s and 75 s. Specifically, the perturbation at 50 seconds is a rapid transient change in the pressure of the cavity, modeled as a Gaussian with a width of 0.5 seconds, centered at 50 seconds, with an amplitude of 1 Torr. The perturbation at 75 seconds is a shift of the pressure of the cavity by 2 Torr, modeled as a tanh function with a width of 0.75 seconds. The transients at these two times are caused by deliberate perturbations to the assumed cavity refractive index, and the main point of the results of FIG. 4 is that there is a rapid return to baseline after these perturbations. In this figure, one can see evidence of the noise in the wavelength measurement, modeled as Gaussian noise with a width of 0.015 FSR.

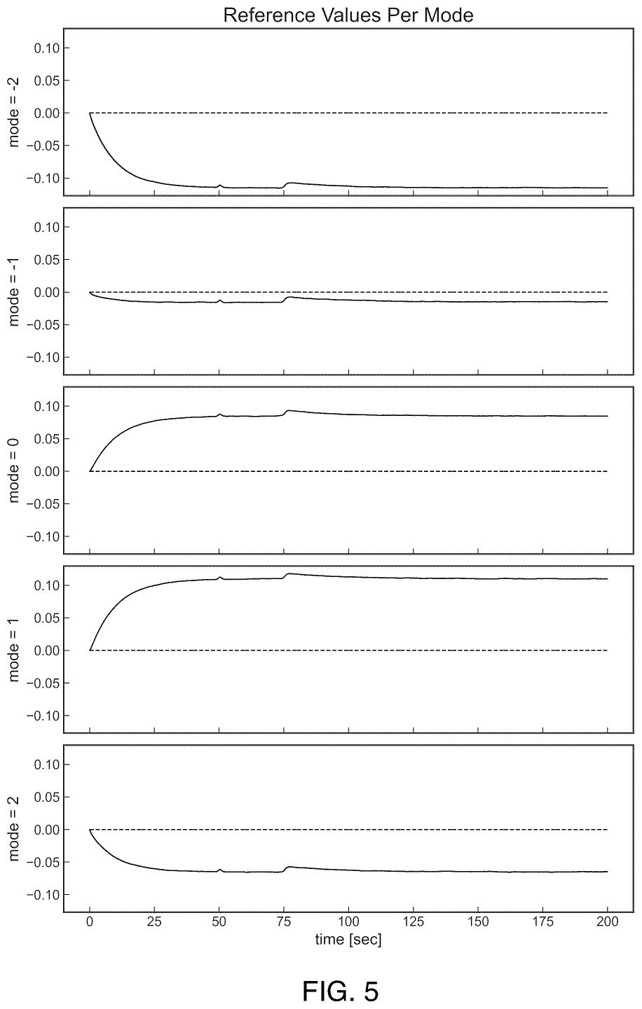

FIG. 5 shows corresponding results for the reference values r[i]. Here it is arg(r[i]) that is plotted, since r[i] itself is complex. The main point of the results of FIG. 5 is that the per-mode reference values largely reflect the static per-mode errors in the wavelength measurement shown in FIG. 3, and that the phase-length perturbations have only small transient effects on these reference values.

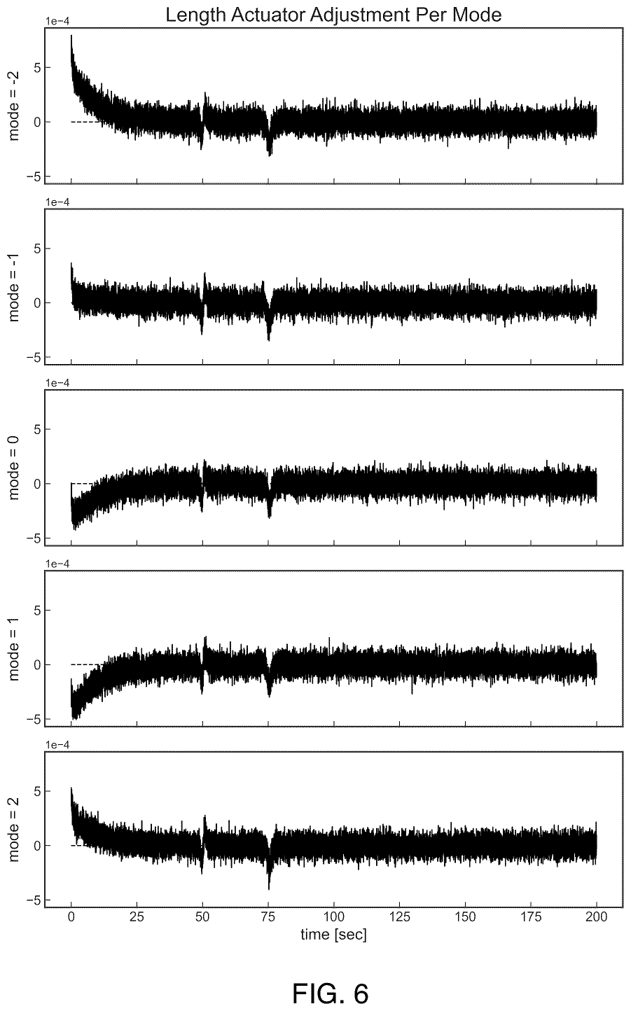

For completeness, FIG. 6 shows the per-mode actuator adjustments, which desirably trend toward zero once the reference values have reached their steady-state values, and are not unduly affected by the perturbations.

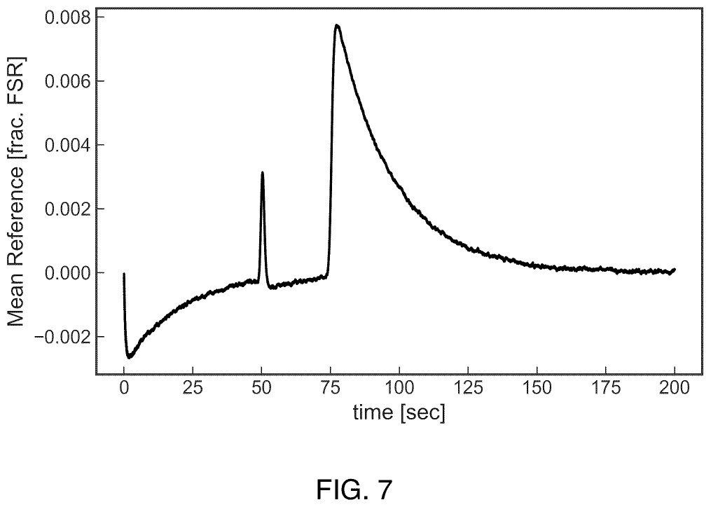

FIG. 7 shows the evolution of the above-described R parameter. Here the effect of the transients is especially apparent.

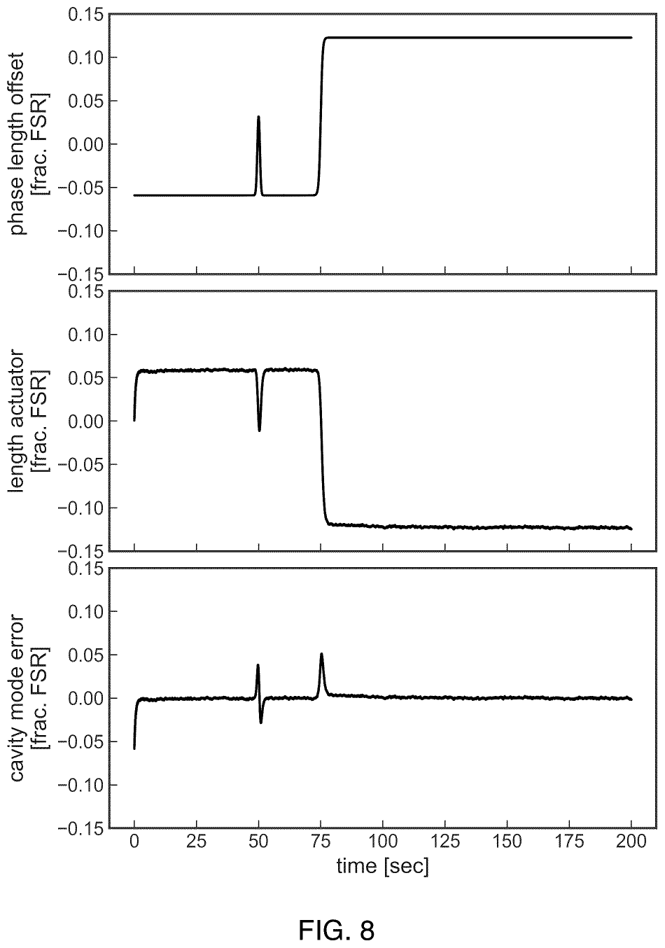

The top two panels on FIG. 8 show compensation between the cavity optical path length (top) and the cavity physical length (middle), with the bottom plot showing the resulting stability of cavity mode errors despite these perturbations.

1. A method of controlling optical path length in a cavity-enhanced spectroscopy system, the method comprising:
generating on-resonance measurements in an apparatus including a laser source, an optical cavity, a frequency monitor and a transducer configured to control an optical path length of the optical cavity;
wherein the on-resonance measurements are generated by tuning an output frequency of the laser source;
at each measurement mode i, computing a frequency difference Δ[i] between a frequency f[i] measured by the frequency monitor and an input target frequency F[i];
at each measurement mode i, automatically adjusting the transducer according to the frequency difference Δ[i] to drive the frequency difference toward zero;
at each measurement mode i, updating a per-mode reference statistic r[i] according at least to a current value of r[i] and Δ[i];
wherein a time constant of the updating the per-mode reference statistic r[i] is slower than a time scale of changes to the optical path length due to changes in gas sample pressure and/or composition;
whereby a frequency comb of the optical cavity is stabilized vs. changes in gas sample pressure and/or composition.

2. The method of claim 1, wherein the cavity-enhanced spectroscopy system is a cavity ring-down spectroscopy system, and wherein the on-resonance measurements are ring-downs.

3. The method of claim 1, wherein the optical cavity has a free spectral range FSR, and wherein a normalized frequency difference w[i] is computed according to w[i]=exp(j2πΔ[i]/FSR), and wherein r[i] is updated according at least to a current value of r[i] and w[i].

4. The method of claim 3, wherein a discrepancy δ[i] is computed according to δ[i]=arg(w[i])−arg(r[i]) mod 2π with −π<δ[i]≤π.

5. The method of claim 4, wherein M is a maximum single-step adjustment, wherein SF is a scale factor, and wherein the transducer adjustment is given by min(M, max(−M, SF δ[i])).

6. The method of claim 3, wherein K is a correction factor, wherein α is a first time constant parameter having an effective time constant τ_{α}, and wherein r[i] is updated according to r[i]←K((1−α)r[i]+αw[i]).

7. The method of claim 6, wherein τ_{α} is in a range between 1 minute and 3 minutes.

8. The method of claim 1, wherein a spectral scan having a scan duration T_{scan} is performed by gathering data at least once for each measurement mode, and wherein τ_{α} is in a range between 10 T_{scan} and 1000 T_{scan}.

9. The method of claim 6, wherein β is a first relaxation factor, and wherein K includes a factor exp(jβδ[i]), whereby an average amount the transducer needs to be adjusted for all measurement modes is minimized.

10. The method of claim 9, wherein β is in a range between 0.05 and 0.5.

11. The method of claim 6, wherein ρ is a second time constant parameter having an effective time constant τ_{ρ}, and wherein an average reference value R is updated at each ringdown according to R*←(1−ρ)R+ρr[i].

12. The method of claim 11, wherein τ_{ρ} is in a range between 30 seconds and 2 minutes.

13. The method of claim 11, wherein τ_{ρ}<0.5 Ta.

14. The method of claim 1, wherein a spectral scan having a scan duration T_{scan} is performed by gathering data at least once for each measurement mode, and wherein τ_{ρ} is in a range between 10 T_{scan} and 100 T_{scan}.

15. The method of claim 11, wherein γ is a second relaxation factor, and wherein K includes a factor exp(−jγarg(R)), whereby arg(R) is minimized.

16. The method of claim 15, wherein γ is in a range between 0.002 and 0.05.

17. The method of claim 1, further comprising locking the frequency comb of the optical cavity to at least one spectroscopic reference.

18. The method of claim 1, further comprising initializing the per-mode reference statistic r[i] to a real value on the order of (machine precision)^{1/2} for each mode.

19. The method of claim 1, wherein the transducer is configured to adjust a physical length of the optical cavity.

20. The method of claim 1, wherein the transducer is configured to adjust a refractive index of the optical cavity via gas flow control.
